(19) United States
(12) Plant Patent Application Publication
(10) Pub. No.: US 2020/0077560 P1
(43) Pub. Date: Mar. 5, 2020

(54) Distinct Variety of Leucadendron Plant Originated From Spontaneous Mutation Within a Cultivated Planting Of Leucadendron Gold Cup

(50) ;

(71) Applicant: Duncan Wood, Yallingup Siding (AU)

(72) Inventor: Duncan Wood, Yallingup Siding (AU)

(21) Appl. No.: 16/602,003

(22) Filed: Jul. 19, 2019

Related U.S. Application Data
(60) Provisional application No. 62/765,221, filed on Aug. 29, 2018.

(51) Int. Cl. A01H 6/36

(52) U.S. Cl. PLT/263.1

(57) ABSTRACT

A distinct cultivar of Leucadendron female plant named “Platinum Cup”, characterized by its production of long stems with pubescent silver leaves and 8 cm silver cup shaped flower heads. The silvery appearance of the cultivar makes it distinct from all other Leucadendrons of similar species, shape and form.

### CROSS REFERENCE TO RELATED APPLICATIONS

Provisional application No. 62/765,221. Filing date Aug. 29, 2018.

Latin name of the genus and species of the plant claimed: Leucadendron laureolum x Leucadendron salignum.

Variety denomination: Platinum Cup.

### BACKGROUND OF THE INVENTION

1. The plant, being a hybrid of Leucadendron laureolum and Leucadendron salignum is a shrub intended for cutflower/cut foliage production and marketed as cutflowers/foliage for florist industry.

2. The parent plant is Leucadendron laureolum x salignum variety Gold Cup and a single branch of this variety within a cultivated planting on the inventor's property at Yallingup Siding, Western Australia was found to have mutated and displayed silver leaves and flower heads instead of the parent plant's green/yellow coloration. Vegetative cuttings were taken from this mutated branch and resultant rooted plants displayed same silver colour when flowering commenced 4 years later. No off types were observed. A further round of cuttings was therefore subsequently taken from these plants and again resultant rooted plants were planted in the field. All of these plants again displayed same silver colour once flowering commenced. No off types were observed.

### SUMMARY OF THE INVENTION

The following traits have been repeatedly observed and represent the characteristics of the new cultivar. These attributes in combination distinguish “Platinum Cup” as a unique cultivar of Leucadendron.

1. “Platinum Cup” exhibits silver colored leaves.

2. “Platinum Cup” exhibits pubescent leaves.

3. “Platinum Cup” exhibits a fringe on leaf margins.

4. “Platinum Cup” exhibits greenish yellow colored outer involucral leaves of flower head.

5. “Platinum Cup” exhibits conspicuous pubescence on inner involucral leaves of flower head.

6. “Platinum Cup” exhibits predominantly yellow green color of the inner involucral leaves.

7. “Platinum Cup” exhibits green colored female floret masses.

8. “Platinum Cup” exhibits predominantly cream colored basal bracts of floret mass.

The parent of “Platinum Cup, “Gold Cup” differs from “Platinum Cup” in having green colored leaves that are not pubescent and have no fringe on margin. The outer involucral leaves of the parent are also bright yellow with red margins. The parent also differs from “Platinum Cup” in not having pubescent inner involucral leaves which are also predominantly yellow in color with red tips. “Gold Cup” also differs from “Platinum Cup” in having pink colored floret masses with yellow basal bracts.

### BRIEF DESCRIPTION OF THE DRAWINGS

The accompanying colored photographs illustrate the overall appearance and distinct characteristics of the new cultivar. The photographs were taken of 3 year old plants of the new cultivar “Platinum Cup” as grown in open field conditions in Yallingup Siding, Australia.

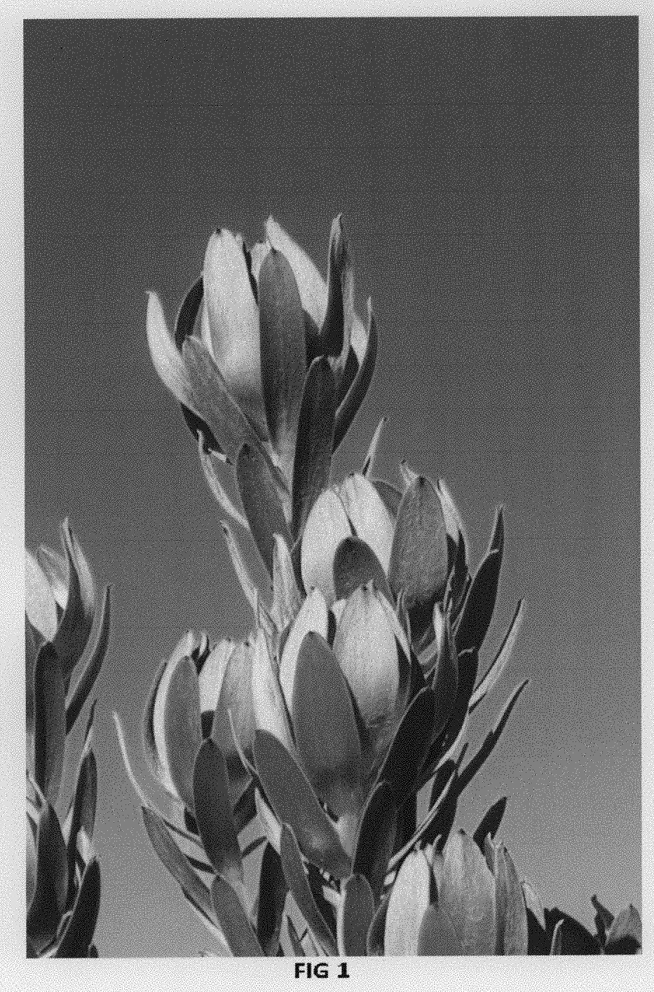

The photograph in FIG. 1 provides a view of the flower heads of “Platinum Cup” in the field.

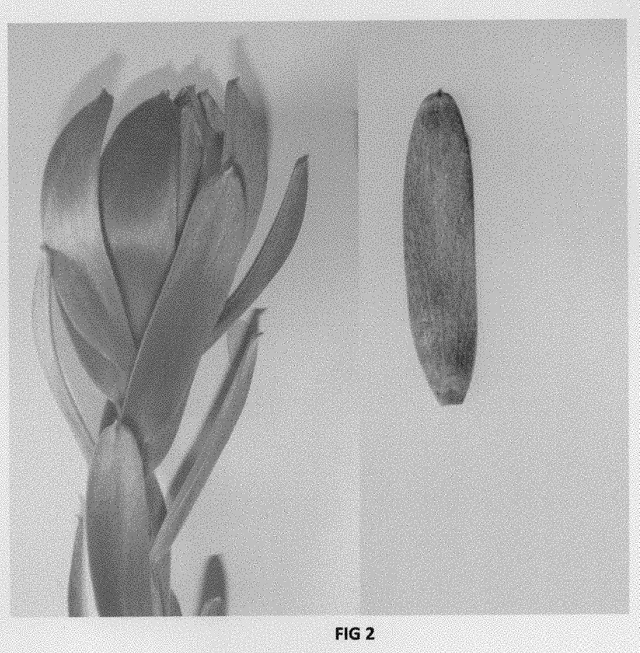

The photograph in FIG. 2 provides a detailed view of a flowering stem and individual leaf of “Platinum Cup”.

The colors in the photographs are as close as possible with digital photography techniques available and the color values cited in the detailed botanical description accurately describe the colors of the new Leucadendron.

### DETAILED BOTANICAL DESCRIPTION OF THE PLANT

The following is a detailed description of three year old plants of the new cultivar as grown in open field conditions at Yallingup Siding, Australia. The phenotype of the new cultivar may vary with variations in environmental, cultural and climatic conditions as it has not been tested under all possible environmental conditions. The color determination is in accordance with The 1986 R.H.S. Color Chart of The Royal Horticultural Society, London, England, except where general color terms of ordinary dictionary significance are used.
General description:
   Blooming habit.—Blooms for 8 weeks in late winter to mid spring in Yallingup Siding, Australia.
   Plant sex.—Female.
   Plant type.—Evergreen shrub.
   Plant habit.—Upright.
   Height and spread.—2.5 m in height and 1.5 m spread.
   Hardiness.—At least in U.S.D.A. zones 9 to 11.
   Diseases.—No resistance or susceptibility to diseases has been observed.
   Environmental stresses.—Grows well in nutrient poor sandy to loam soil, low to medium humidity and hot sun.
   Propagation.—Semi hardwood stem cuttings.
   Growth.—Vigorous.
   Roots.—Strong thick branched primary roots with clusters of proteoid secondary roots.
   Rooting.—6 weeks in average temperature of 24 C. in well drained media in a greenhouse with rooting hormone and spray mist of leaves. Rooted plants fill a 3-inch tube in 2 months at 25 C.
Branch description:
   Stem color.—New growth; green (144B) and rapidly becoming reddish (179B), mature wood; brown (166B).
   Stem surface.—New growth; pubescent, mature wood; smooth and dull.
   Branching.—1 basal branch, 0 to 3 lateral branches per main stem, no tertiary branches.
   Internode length.—An average of 1.5cm in length.
   Stem size.—Basal branch; average 10 mm in width and 80 cm in length, lateral branches if present; up to 20 cm in length and an average of 5 mm in width.
Foliage description:
   Leaves.—Simple, linear in shape, spirally alternate, average of 6.5 cm in length and 1.5 cm in width, internode length average 1.5 cm, attachment sessile, pubescent on both surfaces with a fringe on leaf margin, color: new and mature foliage both surfaces; color of silver (191D). Leaf apex; rounded. Leaf base; obtuse. Leaf in cross section; flat.
Inflorescence description:
   Inflorescence type.—Single on terminals of basal branch and laterals.
   Inflorescence size.—8 cm in length and 8 cm in diameter.
   Inflorescence number.—1 per basal branch or lateral.
   Flower number.—Masses of florets, about 100 per inflorescence.
   Flower fragrance.—None.
   Inflorescence longevity.—About 3 months in field.
   Longevity as cut flower.—3 to 4 weeks.
   Harvest production period for cut flowers.—Up to 3 months.
   Harvest production yield.—50 stems on plants of 3 years or more.
   Flower type.—Cup to goblet shaped leaf bracts enclosing a mass of florets.
   Outer involucral leaf bracts.—Shape; elongated, broad in centre and pointed at apex. Margin; entire. Texture; pubescent. Color; greenish yellow (145C).
   Inner involucral leaf bracts.—Shape; elongated, broad in centre and pointed at apex. Margin; entire. Texture; pubescent. Color; yellow green (150C).
   Basal bracts.—Shape; elongated, broad in centre and pointed at apex. Margin; entire. Texture; pubescent. Number of basal bracts per inflorescence; about 15. Color cream (158A).
   Floral bracts.—The cone of about 100 floral bracts is green (143B).
Reproductive organs:
   Pistils.—In the axil of each floral bract is a floret, which has a cream colored stigma surrounded by anthers.
   Stamens.—Each floret has 4 sterile cream colored anthers which reflex to allow pollination of the single stigma.
Seed production: Seed production has been observed following development of floral cone after pollination by other varieties or species of male Leucadendron plants.

It is claimed:

1. A new and distinct cultivar of Leucadendron plant named “Platinum Cup”, as herein illustrated and described.
